(19) United States
(12) Patent Application Publication
(10) Pub. No.: US 2025/0090765 A1
(43) Pub. Date: Mar. 20, 2025

(54) AUTOINJECTOR WITH ANTI-ACTIVATION FEATURE

(71) Applicant: Owen Mumford Limited, Woodstock (GB)

(72) Inventors: Robert William Bruns, Woodstock (GB); Tahir Shabudin, Woodstock (GB)

(21) Appl. No.: 18/884,689

(22) Filed: Sep. 13, 2024

(30) Foreign Application Priority Data
Sep. 15, 2023 (GB) 2314112.0

(51) Int. Cl. A61M 5/32; A61M 5/20; A61M 5/315

(52) U.S. Cl. CPC A61M 5/3204; A61M 5/31576; A61M 2005/2006

(57) ABSTRACT

An autoinjector comprises a main housing for receiving a syringe or cartridge, a drive mechanism located within the main housing, and a shroud, coupled to a front end of the main housing and movable relative to the housing between an extended position in which a needle tip is substantially shrouded and a retracted position in which the needle tip can extend through an opening in the shroud. The shroud is coupled to the drive mechanism so that movement of the shroud causes a release of the drive mechanism, and the shroud is biased towards the extended position. A device cap and the shroud comprise respective substantially inflexible formations which are directly mechanically engageable with one another to prevent or restrict inward movement of the shroud relative to the housing when the cap is attached to the housing.

### TECHNICAL FIELD

The present invention relates to autoinjectors for drug delivery and provided with an anti-activation feature to prevent or reduce the risk of accidental activation.

### BACKGROUND

Mechanically powered autoinjectors are commonly used to deliver many different types of drug. In the majority of injectors, the force to deliver an injection is provided by powerful helical springs, either compressed and released to provide the delivery force or expanded and contracted to provide that force. In some cases, the same or a different spring mechanism provides a needle insertion force to cause a syringe or cartridge needle tip to penetrate a user's skin, prior to drug delivery.

WO2022/179832 and WO2020/064927 describe autoinjectors suitable for use with safety syringes, for example as described in WO2019/086718. These autoinjectors comprise a base portion for receiving a safety syringe and a lid portion hingeably connected to the base portion. The action of opening and closing the lid portion to load a syringe acts to prime a pair of main drive springs. To fire the loaded and primed autoinjector, the user urges a shroud at a front end of the autoinjector into contact with an injection site (e.g., a user's skin), releasing the drive springs and forcing the drug through the syringe needle and into the injection site.

In striving to improve reliability, manufacturers of autoinjectors seek to identify events that might give rise to accidental firing, e.g. or a primed and loaded autoinjector.

WO2019/141985 describes an autoinjector with a removable cap and locking member for preventing accidental activation. The locking member is movable within the removable cap such that the cap is a two-part component.

### SUMMARY

According to the present invention there is provided an autoinjector for delivering a dose of medication into a patient from a medication containing syringe or cartridge having a needle affixed thereto. The autoinjector comprises a main housing for receiving the syringe or cartridge, a drive mechanism located substantially within the main housing for providing motive force to deliver medication into the patient from the syringe or cartridge, and a shroud defining an opening therethrough, coupled to a front end of the main housing and movable relative to the housing between an extended position in which a needle tip is substantially shrouded and a retracted position in which the needle tip can extend through the opening in the shroud. The shroud is coupled to the drive mechanism so that movement of the shroud from the extended position to the retracted position causes or permits a release of the drive mechanism to provide said motive force, and the shroud being biased towards the extended position. The cap is configured for removable attachment to a front end of the housing to shield the shroud and thereby prevent user contact with the shroud prior to removal of the cap. Furthermore, the cap and the shroud comprise respective substantially inflexible formations, the inflexible formations being directly mechanically engageable with one another, when the cap is attached to the housing, to prevent or restrict inward movement of the shroud relative to the housing when the cap is attached to the housing.

The inflexible formations may depend from the cap and the shroud and overlap in a direction transverse to an axis of the autoinjector along which said motive force is directed. The inflexible formations may be integral with the cap and the shroud from which they depend. Optionally the inflexible formations may define respective opposed surfaces with a same or similar non-perpendicular slope relative to said direction.

The housing may comprise first and second main housing parts movable relative to one another between an open position in which the syringe or cartridge can be inserted into a receiving slot defined within the first main housing part and a closed position in which the inserted syringe or cartridge is securely retained in the receiving slot and within the housing. The shroud comprises a first shroud part coupled to the first main housing part and a second shroud part coupled to the second main housing part such that the shroud parts come together when the main housing parts are in the closed position to form a substantially unitary shroud, and the shroud being coupled to the drive mechanism by the first shroud part and the first main housing part.

The formation on the shroud may be a formation on the first shroud part. The cap and the first shroud part may each comprise a substantially rigid one-piece or multi-piece moulded plastic component, and the respective inflexible formations may be moulded integrally with one or more of the pieces.

The cap and the main housing may comprise respective cooperating features facilitating a snap-fit engagement of the cap and the main housing, said inflexible formations defining opposed contact surfaces that are spaced apart by a distance sufficient to enable snap-fit engagement and disengagement of the cap and the housing after and before axial alignment of the contact surfaces.

### BRIEF DESCRIPTION OF THE DRAWINGS

FIG. 1A-C show an autoinjector in a (A) closed, (B) partially open and (C) open state;

FIG. 2 shows a safety syringe;

FIG. 3 shows a capped end of the autoinjector of FIGS. 1A-C;

FIGS. 4A and 4B show partial cross-sections of the autoinjector of FIGS. 1A-C;

FIG. 5A illustrates schematically a modification to the autoinjector of FIGS. 1A-C, FIG. 3 and FIGS. 4A and B and which provides an anti-activation feature;

FIG. 5B illustrates and enlarged detail of the modification of FIG. 5A;

FIG. 6A illustrates a cap of the autoinjector of FIG. 5A; and

FIG. 6B illustrates a shroud part of the autoinjector of FIG. 5A.

### DETAILED DESCRIPTION

In seeking to improve the reliability of autoinjectors and in particular to minimise a risk of accidental firing of a loaded and primed auto-injector, the inventors have recognised that autoinjectors of known type, such as those described in WO2022/179832 and WO2020/064927, may be fired accidentally if subjected to a sudden impact. This might occur for example when an autoinjector is dropped on its rear end, i.e. the end opposite the injection end, after a device has been loaded and primed, and can occur even when a removable cap, shielding the injection end, is in place.

The terms “forward” or “front” are used here to refer to the needle side or injection site end of the autoinjector, whereas the term “rear” refers to the end of the autoinjector remote from the needle or injection site.

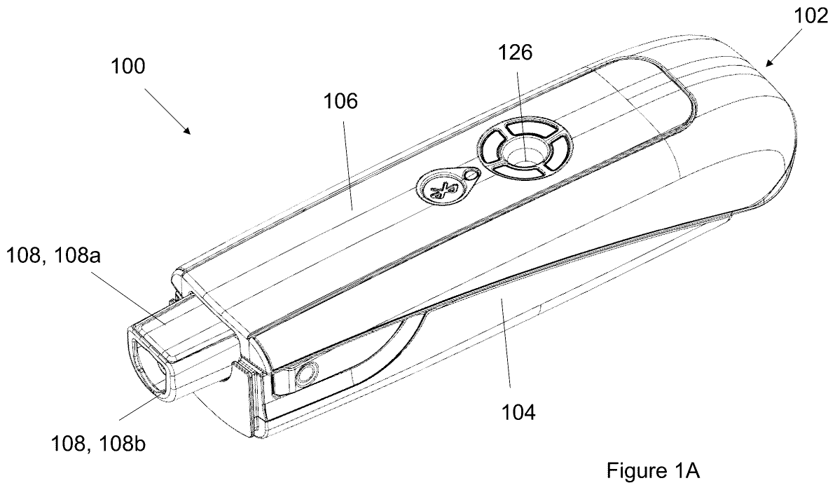

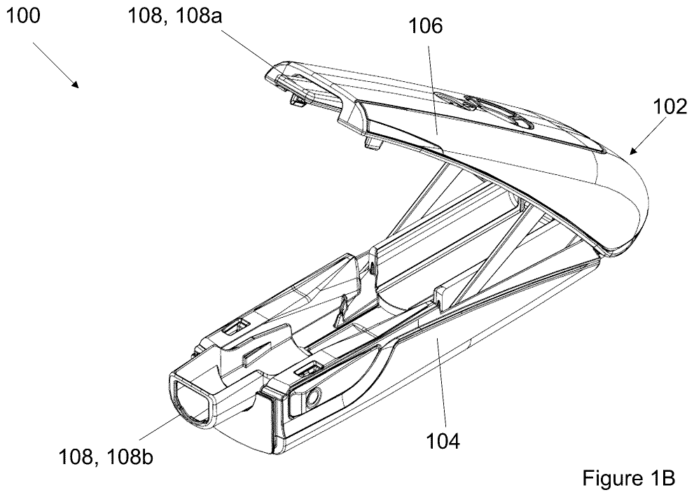

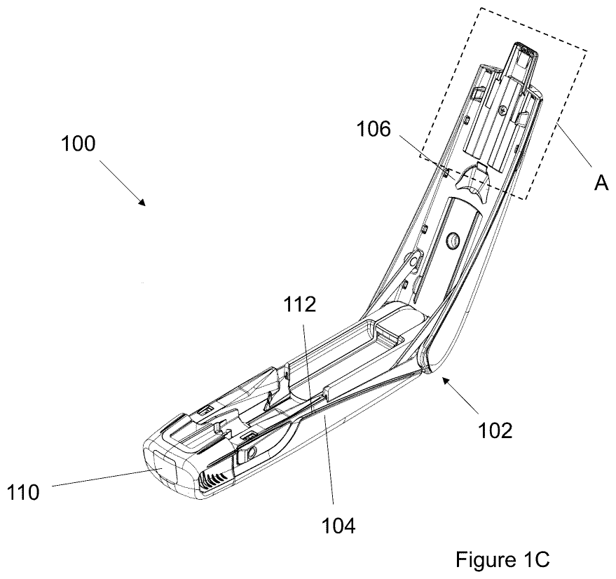

FIGS. 1A-C show an embodiment of a known autoinjector 100 in: A) a closed state; B) a partially open state; and C) a fully open state.

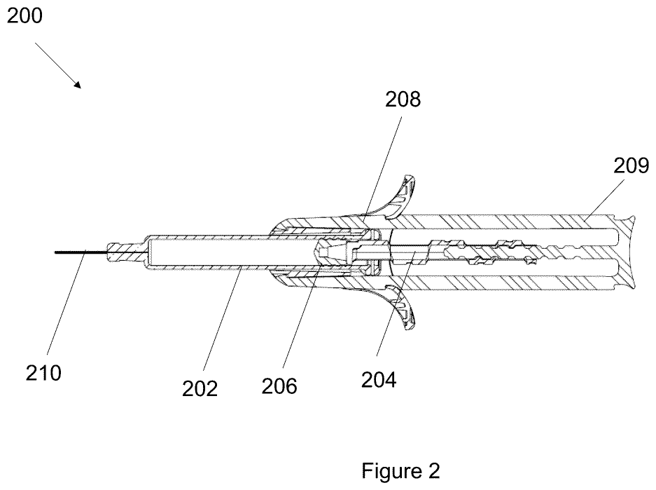

The autoinjector 100 comprises a housing 102 which includes a main body 104 and a lid 106 that are hingedly connected so as to permit opening and closing of the housing. The autoinjector further comprises a plurality of component parts contained within the housing. A syringe such as the syringe 200 of FIG. 2 (not shown in FIGS. 1A-C) is receivable within the housing in a slot 112 defined in the main body. The lid 106 of the autoinjector 100 includes a through-hole 126 positioned so that a surface of a plunger driver 116, the operation of which is described below, is viewable once firing is complete. That surface is vividly coloured in contrast to other parts visible through the through-hole prior to and during drug delivery to thereby provide a visual indication to the user that drug delivery is complete.

As shown most clearly in FIGS. 1A and 1B, the autoinjector 100 further comprises a shroud 108, formed from a lower part 108a and an upper part 108b. The lower and upper parts are respectively coupled to the main body 104 and lid 106 so that the parts 108a, 108b separate as the housing 102 is opened to allow insertion of a syringe 200 and come together to form the unitary shroud 108 when the housing is closed. The shroud 108 defines an aperture through which the needle 210 of a syringe 200 at least partially extends, when a syringe is received in the autoinjector 100. The lower part and upper part 108a, 108b comprise slidable connections with the main body 104 and lid 106 respectively, to permit movement between an extended position where the end of the syringe needle is substantially covered by the shroud, and a retracted position where the end of the syringe needle is exposed. The shroud parts are separately biased towards the extended position so that the needle of a syringe in the autoinjector remains substantially covered prior to an injection.

The housing parts and the shroud parts may be formed of plastic, e.g. as moulded plastic parts.

As shown in FIG. 1C, the autoinjector 100 includes a removable cap 110 which is normally in place prior to an injection being performed. For ease of understanding, the cap is omitted from FIGS. 1A and 1B. In the configuration shown, the cap fits slidably over the lower shroud part 108b and further abuts against a front end of the main body 104. The cap 110 is intended to prevent user access to the shroud and hence accidental firing while the cap is in place. The cap may be formed of a substantially rigid plastic. It may be a single moulded piece, or may be formed of two or more plastic parts. The cap may also comprise metals fingers as described below.

FIG. 2 illustrates a safety syringe 200 suitable for use with the described autoinjector 100. Such a syringe 200 is described in detail in WO2019/086718. It is sufficient here to note that the syringe comprises a syringe body 202 for containing a medicament, a syringe plunger 204 that engages with a bung 206 within the syringe body, a needle shield 208 coupled to a safety plunger 209, and a needle 210. The coupling between the syringe plunger and the safety plunger/needle shield is such that the needle shield 208 is deployed around the needle of the syringe so as to substantially cover the needle following delivery of the medicament from the syringe body. This coupling is described in detail in WO2019/086718.

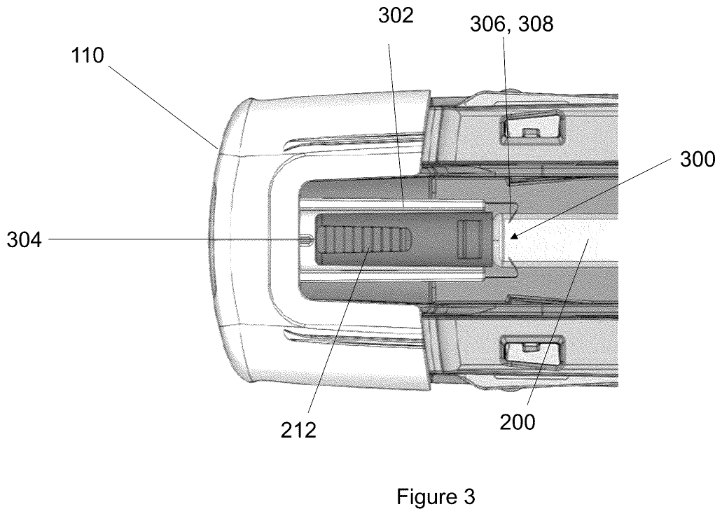

In general, syringes, including safety syringes, are routinely provided with a protective rigid needle shield (RNS) which require removal before a syringe can be used (the RNS is not shown in FIG. 2). To this end, the cap 110 also operates, in a known way, as an RNS remover 300. FIG. 3 shows a top plan view of the end of the autoinjector with the cap in place. The autoinjector 100 is in the open state such that the RNS remover and the end of a syringe 200 with an attached RNS 212 are visible. The RNS remover comprises a U-shaped wall 302 extending away from the cap, with the wall open at its upper side, and which defines a passageway 304 for receiving the RNS when the cap is fitted to the autoinjector. The front end of the side wall terminates with a gripping member 306. The gripping member is configured to allow easy insertion of the RNS 212 into the passageway whilst preventing its withdrawal thereafter. The RNS can therefore be removed from the syringe as the cap is removed. It will be appreciated that the RNS 20 remover 300 (as part of the cap 110) is configured so as to fit and slide within the shroud 108.

In the configuration shown in FIG. 3, the gripping member 306 comprises projections 308 which extend inwardly into the passageway 304, angled away from the front end the side wall 302. The gripping members 306 may be provided by flexible metal legs fixed within the cap 110. As the syringe 200 with RNS is inserted into the slot 112 of the main body 104, the projections 308 are able to flex outwardly, whereas any return motion is prevented by the projections 308 as they come into engagement with the RNS 212.

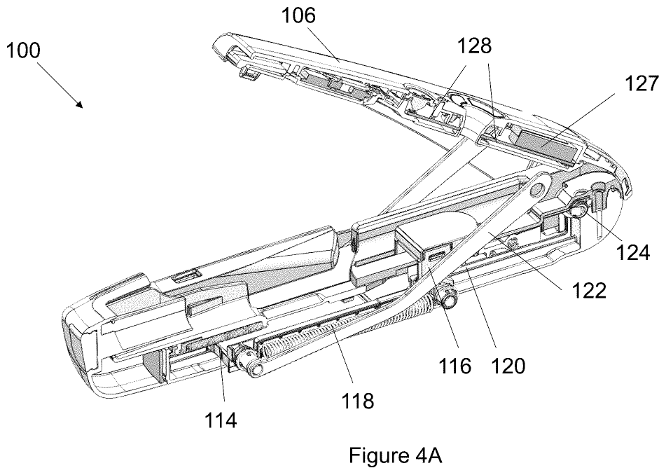

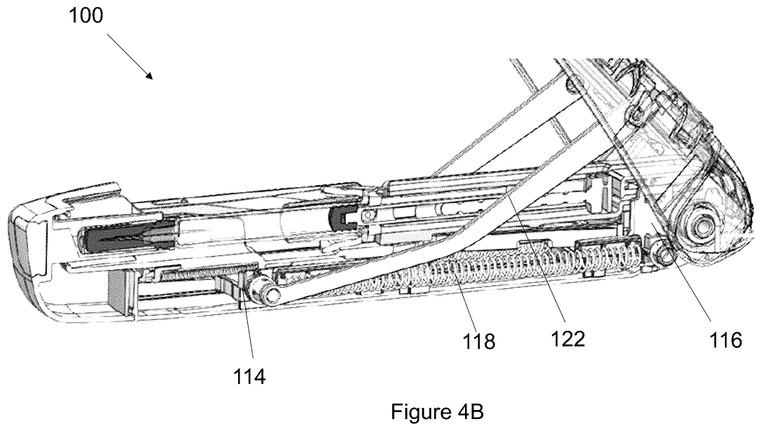

FIGS. 4A and 4B show partial cross-sectional views of the autoinjector 100 during various stages of priming to illustrate the presence and operation of further internal components during lid 106 opening and closing strokes. In particular, it can be seen that the autoinjector 100 comprises a shuttle 114 which is operable to move between a first forward position and a second rearward position along a shuttle guide 120 on the main body 104 of the housing 102, a plunger driver 116 for driving the syringe plunger 204, and a biasing element 118 which couples the shuttle and plunger driver 116. The shuttle and plunger driver are slidably connected to the shuttle guide 120 to permit rearward and forward movement within the housing 102. Unlike the plunger driver, the shuttle is also fixedly connected to the lid 106 via two arm members 122. The plunger driver comprises a rearmost member 117, referred to here as a “push member”, that is located in use behind the rear end of the plunger of an inserted syringe.

The biasing element 118 comprises two extension springs on either side of the device, although only one is visible in the drawings. Prior to any priming, these springs are under slight tension so as to hold the plunger driver 116 and shuttle 114 together. It should therefore be noted that priming, in the context of the extension springs, refers to the process of further tensioning the extension springs into a state whereupon firing can be initiated.

Each of the shuttle guide 120 and the plunger driver 116 comprise part of a latching arrangement, which are configured to cooperate to secure the plunger driver at the rear end of the autoinjector 100. A suitable latching arrangement is described in WO2022/179832.

The autoinjector 100 further comprises a torsion spring 124 arranged at the hinged connection between the lid 106 and main body 104 of the autoinjector 100. The torsion spring is coupled to both the lid and main body. In the embodiment shown, one end of the torsion spring is attached to the lid, and the opposing end is attached to the main body of the autoinjector.

Priming of the autoinjector on the lid opening stroke (FIG. 4A) and the lid closing stroke (FIG. 4B) is now described. WO2021058474 describes operation of a similar autoinjector, except that the biasing element described therein further includes a compression spring.

As the lid 106 is opened, the arm members 122 which couple the lid and shuttle 114 together cause the shuttle to move rearwards from the first position to the second position. The shuttle is in constant engagement with the plunger driver 116 so that its rearward travel causes the same rearward travel for the plunger driver. The extension springs coupled between them therefore remains un-primed (i.e. further extended) during lid opening. Near the end of lid opening stroke, the latching arrangement part on the shuttle guide 120 and the plunger driver are brought together such that they are able to cooperate to secure the plunger driver at the rear end of the autoinjector 100.

Lid 106 opening also causes the end of the torsion spring 124 attached to the lid to rotate about its spring axis relative to the opposing end of the torsion spring. This primes the torsion spring on lid opening. When primed, the torsion spring produces a restoring force which tends to urge the lid closed.

Upon closing of the lid 106, whilst the shuttle 114 is free to move forwards along the shuttle guide 120 to the first position, the plunger driver 116 is held at the rear of the autoinjector by the latching arrangement. Thus, during the lid closing stroke, the shuttle and plunger driver separate and the extension springs coupled between them are primed (i.e., further tensioned).

As has already been noted above, the primed torsion spring 124 urges the lid 106 closed. This assists in priming the extension springs 118 during closing, whilst requiring a minimal force to prime the torsion spring during opening. This is important for users of autoinjectors who would otherwise find it difficult to apply the necessary force to close the lid.

Firing of the autoinjector is now described. The firing mechanism is described in more detail in WO2022179832.

To fire the loaded and primed autoinjector, the user urges the front end of the autoinjector 100 into contact with an injection site (e.g., a user's skin). This causes the shroud parts 108a, 108b to move into the retracted position against their biases (e.g., respective springs). As the shroud retracts into the housing 102, the lower shroud 108b permits or causes release of the latching arrangement and the primed extension springs 118a, 118b. The restoring force of the extension springs, acting on the plunger driver 116, drives the plunger driver, and specifically the push member 117, forwards to depress the syringe plunger and force the drug from out of the syringe needle into the injection site.

It has been found that, in certain instances, a loaded and primed autoinjector having the general structure described can be accidentally fired even when the removable cap 110 is in place. This can happen when the autoinjector is dropped on its rear end. It should be noted that autoinjectors are often used by persons with limited mobility and as such occasional dropping of the autoinjector can be expected. The cause of such accidental firing is suspected to be due to reaction forces which drive the shroud, particularly the lower shroud part 108b, inward relative to the housing 102. According to current designs the cap 110 provides no dedicated mechanism to prevent the risk of such inward movement of the shroud 108. Whilst WO2019/141985 may point to one solution, this is disadvantageous as it requires the manufacture and insertion of a further component within the cap.

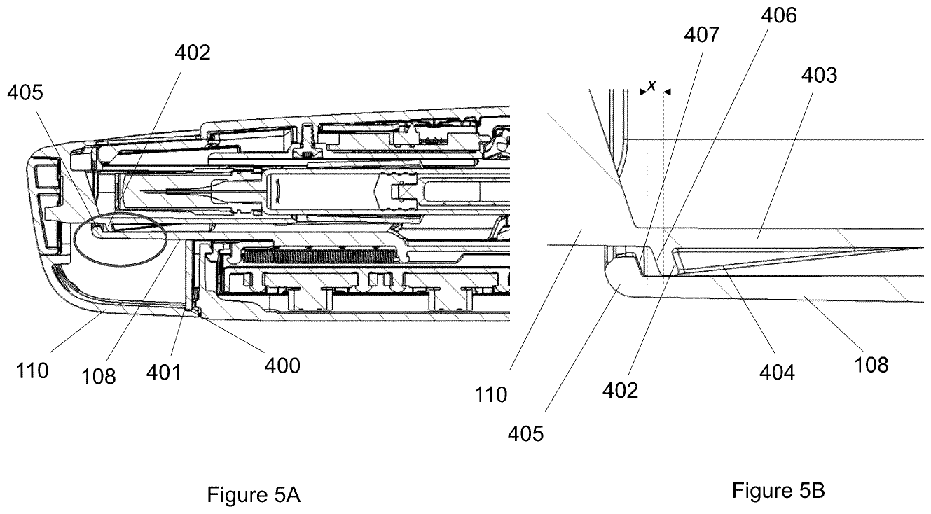

A simple and elegant solution is illustrated in FIGS. 5A, and in FIG. 5B which is an enlarged view of the detail B of FIG. 5A. This is a specific example of a means to latch the removable cap 110 and the shroud 108 together to effectively prevent unintended rearward movement of the shroud whilst the cap is in place. The cap 110 defines a slot 400 on an internal surface extending laterally across a region of the cap. The slot is configured to cooperate with a lip 401 formed at a front edge of the main body 104 of the housing 102 such that, when the cap is pushed onto the front of the housing, the lip 401 snaps into the gap behind the slot 400, holding the cap firmly, but releasably, in place. Further slots and lips may be provided on the sides of the cap and main housing (front part) to assist in securing the cap in place, although these are not shown in the drawings.

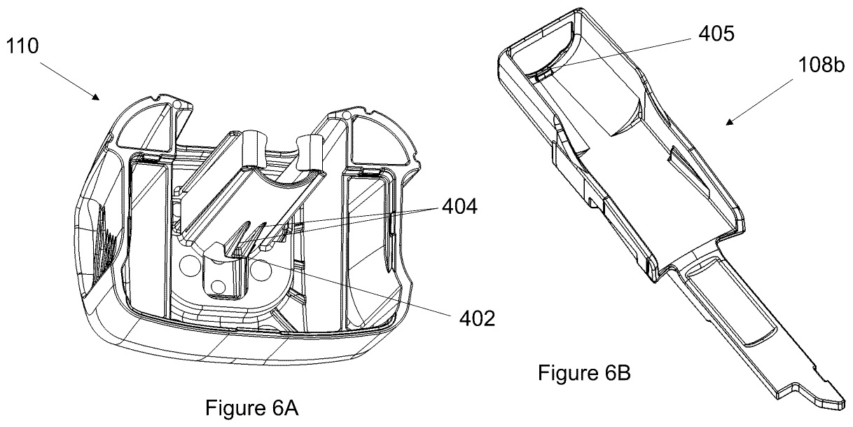

A catch 402 protrudes downwardly from a base 403 of the RNS remover (U-shaped wall 302) part of the cap. The catch is supported by a pair of legs 404 as best shown in FIG. 6A. As is apparent from FIGS. 5A, 5B and 6B, the lower shroud part 108b is provided, proximal to its front end, with a shoulder 405. Both the catch 402 and the shoulder 405 are moulded integrally with the cap and shroud parts from which they depend and are substantially inflexible with respect to those parts. Specifically, the catch and the shoulder provide for no axial movement, and preferably no transverse movement with respect to the parts to which they are attached.

As the cap 110 is initially fitted onto the housing 102 and over the shroud 108, and prior to the snap-fit engagement of the slot(s) 400 and cooperating lip(s) 401, the cap and the lower shroud 108b can move relative to one another, sufficient to allow the ends of the catch 402 and the shoulder 405 to pass over one another. Once this has occurred, further movement of the cap 110 towards the housing 102 causes snap-fit engagement of the slot(s) 400 and cooperating lip(s) 401. Once the cap 110 is snapped in place onto the housing 102, these two parts are substantially rigidly connected.

Consider now an event, such as an accidental dropping of the device onto its rear end, which drives the shroud 108, and in particular the lower shroud part 108b, rearwards into the main housing. After only a very small movement of the lower shroud part, the shoulder 405 will hit the catch 402. As both features are inflexible, any further rearward movement of the lower shroud part 108b into the housing will be blocked. This direct mechanical contact between opposed faces of the shoulder and the catch will thereby prevent accidental firing of the device whilst the capo is in place.

When a user elects to remove the cap 110 from the housing 102 to expose the shroud 108 prior to use, pulling of the cap initially disengages the cap from the housing. FIG. 5B illustrates with legend “x” the relative movement of the cap and the shroud that is permitted before the catch 402 and the shoulder 405 come into contact. This distance is sufficient to allow for disengagement of the snap-fit coupling between the cap and the housing. Following this disengagement, the cap can be tilted by a user relative to the housing to allow the ends of the catch and the shoulder to pass over one another. The cap 110 can then be completely removed.

In order to better facilitate complete disengagement of the cap and the housing, opposed surfaces 406,407 of the catch 402 and the shoulder 405 may be arranged at an angle offset from a direction that is transverse to the axis of the device, i.e. the surfaces may be sloping. The angled surfaces encourage tilting of the cap relative to the shroud to facilitate disengagement. The angle relative to the transverse direction may be between 5 and 45 degrees.

The skilled reader will be able to envisage further embodiments of the invention without departing from the scope of the appended claims. For example, rather than providing cooperating formations as a first lip extending upwardly from the lower shroud part and a second lip extending downwardly from a base of the RNS remover, features may be provided on a lower surface of the lower shroud part and on a part of the cap beneath that lower shroud part. Other configurations will be readily apparent.

1. An autoinjector for delivering a dose of medication into a patient from a medication containing syringe or cartridge having a needle affixed thereto, the autoinjector comprising:
a main housing for receiving the syringe or cartridge;
a drive mechanism located substantially within the main housing for providing motive force to deliver medication into the patient from the syringe or cartridge;
a shroud defining an opening therethrough, coupled to a front end of the main housing and movable relative to the housing between an extended position in which a needle tip is substantially shrouded and a retracted position in which the needle tip can extend through the opening in the shroud, the shroud being coupled to the drive mechanism so that movement of the shroud from the extended position to the retracted position causes or permits a release of the drive mechanism to provide said motive force, and the shroud being biased towards the extended position; and
a cap configured for removable attachment to a front end of the housing to shield the shroud and thereby prevent user contact with the shroud prior to removal of the cap,
wherein the cap and the shroud comprise respective substantially inflexible formations, the inflexible formations being directly mechanically engageable with one another, when the cap is attached to the housing, to prevent or restrict inward movement of the shroud relative to the housing when the cap is attached to the housing.

2. An autoinjector according to claim 1, wherein said inflexible formations depend from the cap and the shroud and overlap in a direction transverse to an axis of the autoinjector along which said motive force is directed.

3. An autoinjector according to claim 2, wherein said inflexible formations define respective opposed surfaces with a same or similar non-perpendicular slope relative to said direction.

4. An autoinjector according to claim 1 wherein the inflexible formations are integral to their respective cap and shroud.

5. An autoinjector according to claim 1, the housing comprising first and second main housing parts movable relative to one another between an open position in which the syringe or cartridge can be inserted into a receiving slot defined within the first main housing part and a closed position in which the inserted syringe or cartridge is securely retained in the receiving slot and within the housing,
wherein the shroud comprises a first shroud part coupled to the first main housing part and a second shroud part coupled to the second main housing part such that the shroud parts come together when the main housing parts are in the closed position to form a substantially unitary shroud, and
the shroud being coupled to the drive mechanism by the first shroud part and the first main housing part.

6. An autoinjector according to claim 5, wherein said formation on the shroud is a formation on the first shroud part.

7. An autoinjector according to claim 5, wherein said cap and said first shroud part each comprise a substantially rigid one-piece or multi-piece moulded plastic component, and the respective inflexible formations are moulded integrally with one or more of the pieces.

8. An autoinjector according to claim 1, the cap and the main housing comprising respective cooperating features facilitating a snap-fit engagement of the cap and the main housing, said inflexible formations defining opposed contact surfaces that are spaced apart by a distance sufficient to enable snap-fit engagement and disengagement of the cap and the housing after and before axial alignment of the contact surfaces.
